(19) United States
(12) Patent Application Publication
(10) Pub. No.: US 2008/0254112 A1
(43) Pub. Date: Oct. 16, 2008

(54) Pharmaceutical Active-Ingredient-Containing Formulation with Coating

(76) Inventors: Karin Klokkers, Feldkirchen-Westerham (DE); Marion Zellner, Egrnating (DE); Thomas Rillman, Bellheim (DE); Andreas Dauer, Heufeld (DE)

Correspondence Address: Ronald R. Santucci;Frommer Lawrence & Haug, 745 Fifth Avenue, New York, NY, 10151, US

(21) Appl. No.: 10/577,569

(22) PCT Filed: Oct. 28, 2004
(86) PCT No.: PCT/EP04/12230

§ 371 Date: Feb. 27, 2008

(30) Foreign Application Priority Data
Oct. 31, 2003 (DE) 103 51 301.9
Mar. 24, 2004 (DE) 10 2004 014 828.7

(51) Int. Cl. A61K 9/32; A61K 47/32; A61K 47/30; A61K 9/14; A61K 9/48

(52) U.S. Cl. 424/456; 514/772.3; 514/772.6; 424/497; 424/451; 424/482

(57) ABSTRACT

The invention relates to a pharmaceutical active-ingredient-containing formulation for oral administration which is coated with a single coating of a film-forming polymer, the coating comprising a mixture of at least two separating agents and no stabilizer.

The invention relates to an active-ingredient-containing formulation for oral administration which is coated with a film-forming polymer.

Oral administration of active ingredients exhibits good patient compliance. An appropriate pharmaceutical formula-tion, for example a modified-release medicament form, is developed in accordance with the properties of the active ingredient and the desired release profile. Such forms include delayed-release or retarded-release formulations.

For the preparation of delayed-release medicament forms it is possible for tablets or pellets to be coated with enteric films which are soluble in the small intestine.

In the case of retard formulations, a distinction is made between matrix systems, wherein the active ingredient is mixed with a retarding matrix (polymer, wax), and reservoir systems, wherein an active-ingredient-containing core (e.g. a tablet or pellet) is coated with a polymer film. The retarding coating encapsulates the active ingredient and thus allows gradual release.

As retard formulations there are preferably used multiple-unit-dosage forms. A multiple-unit-dosage form may be a tablet which rapidly disintegrates in the stomach and releases a large number of coated units (pellets). It may also be in the form of a capsule filled with pellets.

An advantage of a retard formulation is the uniform and sustained effective active ingredient level. The time interval between individual tablet ingestions is greater in the case of retard medicament forms than in the case of rapid-release formulations. It is thus possible to achieve better patient compliance.

Advantages of a multiple-unit-dosage form are:
reduced risk of “dose dumping”
dispersed active ingredient dose, that is to say the risk of local irritation is low
in vivo behaviour: only slight variations in release in the stomach, so that reproducible absorption of active ingredient is ensured
the small pellets can be mixed with food and thus facilitate ingestion, especially for elderly patients

Polymer coatings hitherto have in most cases been sprayed onto the pellets or tablets from organic solution. On environmental grounds, however, it is necessary to change over to water-based film-forming polymers.

Polyacrylates are suitable as water-based film-forming polymers.

The term “polyacrylate” is used to denote polymers based on acrylic acid, methacrylic acid, acrylate esters and/or meth-acrylate esters. Polyacrylates are obtainable under the trade names “Eudragit” from Röhm and “Kollicoat” from BASF.

In the literature, the following methods are described for coating pellets or tablets with aqueous polymer dispersions.

In accordance with EP 0 403 959 A1 and also “Glyceryl mono-stearate as a glidant in aqueous film-coating formulations”, H.-U. Petereit et al., Eur. J. Pharm. 41 (4) 219-228, (1995), cores (pellets, tablets) are coated with aqueous acrylate-based polymer dispersions using lipophilic emuls-ifiers such as glycerol monostearate which prevent the pellets from sticking together and which form stable aqueous dispersions with the polymers.

In “Coating of Ibuprofen crystals with Eudragit FS30D”, S. Schmid, Arch. Pharm. Med. Chem. 333, Suppl. 1, 2000, 1-40, No. 78, the particles are prevented from sticking together by adding glyceryl, monostearate as separating agent and polysorbate 80 as wetting agent to the Eudragit FS30D polymer dispersion. The film quality is dependent upon the proportion by weight of glyceryl monostearate and poly-sorbate.

“Microencapsulated Eudragit RS30D coated controlled-release pellets: the influence of dissolution variables and topo-graphical evaluation”, T. Govender, J. Microencapsulation, Vol. 14, No. 1, 1997, pp. 1-13, describes the use of magnesium stearate as “anti-sticking agent” for the coating of cores (diameter about 1.9 mm) with Eudragit RS30D. The release of active ingredient is based on first-order kinetics.

U.S. Pat. No. 5,529,790 discloses the achievement of a specific release rate of active ingredient pellets by the use of aqueous polymer dispersions such as Eudragit NE or Eudragit RS with additives. Additives used for controlling permeability are especially magnesium stearate in combination with citric acid or Simethicone. Magnesium stearate also prevents agglomeration of the cores (500 to 1500 microns) during coating.

In “Eudragit RL and RS Pseudolatices: properties and performance in pharmaceutical coating as a controlled release membrane for theophylline pellets”, R.-K. Chang, Drug Development and Industrial Pharmacy, 15 (2), 1989, pp. 187-196, talcum and silicon dioxide are used as separating agents for coating theophylline pellets with Eudragit RL and/or RS pseudolatices. Organic solvents are also used for the preparation of the Eudragit dispersion.

“Effect of application temperature on the dissolution profile of sustained-release theophylline pellets coated with Eudragit RS30D”, P. Schmidt, F. Niemann, Drug Develop-ment and Industrial Pharmacy, 19 (13), 1603-1612 (1993), describes the coating of theophylline pellets (100 to 1400 m) in a “miniature fluid-bed pan coater” with Eudragit RS30D, various plasticizers (triethyl citrate, dibutyl phthalate, PEG) and talcum.

“Drug release from compressed Eudragit RS30D coated beads”, G. F. Palmieri, S.T.P. Pharma. Sciences 6 (2), 1996, pp. 118-121, relates to the coating of theophylline-containing cores (diameter 200 to 630 m) with Eudragit RS30D, 20% triethyl citrate (plasticizer) and 50% talcum (based on the dry weight of the polymer), which is used for reducing the stickiness of the Eudragit RS30D. Capsules filled with the active ingredient granules exhibit zero-order release, and granules compressed to form tablets exhibit zero-order to first-order release according to granule content.

“Influence of plasticizer concentration and storage conditions on the drug release rate from Eudragit RS30D film-coated sustained release theophylline pellets”, K. Amighi, A. Moes, Eur. J. Pharm. Biopharm. 42 (1) 29-35 (1996) describes the coating of theophylline pellets (diameter 1100 m) with Eudragit RS30D using HPMC, talcum, triethyl citrate (plasticizer) and also silicone emulsion (antifoam).

In “Effect of pectinolytic enzymes on the theophylline release from pellets coated with water insoluble polymers containing pectin HM or calcium pectinate”, R. Semde, Int. J. Pharm., 197, 2000, pp. 169-179, and also in “Influence of curing conditions on the drug release rate from Eudragit NE30D film coated sustained-release theophylline pellets”, K. Amighi, S.T.P. Pharma Sciences 7 (2), 1997, pp. 141-147, talcum and a silicone emulsion (antifoam) are used as auxiliaries in the coating of pellets (diameter 1 mm) with Eudragit NE30D, the film also containing pectin. Eudragit RS30D is processed using a silicone emulsion.

“A pH-dependent colon targeted oral drug delivery system using methacrylic acid copolymers”, M. Z. I. Khan, Journal of Controlled Release 58 (1999), 215-222, describes the coating of tablets with Eudragit S100 and Eudragit L 100-55 or mixtures thereof comprising an aqueous dispersion with triethyl citrate as plasticizer and talcum as glidant.

“Modifying the release properties of Eudragit L30D”, N. A. Muhammad, et al., Drug development and industrial pharmacy, 17(18), 2497-2509 (1991), describes the coating of active-ingredient-containing cores with an aqueous dispersion of Eudragit L30D, triethyl citrate and kaolin in a Glatt GPCG3 with subsequent “curing” of the pellets at 45° C.

Us 20020160046 describes a retard formulation for omeprazole, omeprazole-containing cores being provided with a “thick” retarding coating (100 to 5000 microns), for example containing non-enteric forms of Eudragit. Omeprazole cores are sprayed with an aqueous dispersion of Eudragit NE30D, talcum, magnesium stearate, glycerol monostearate and triethyl citrate. The coating in this case contains the surfactant glycerol monostearate.

WO 99/12524 describes the preparation of multiple-unit-dosage forms for NSAIDs. Active-ingredient-containing cores are coated, for example, with a mixture of Eudragit NE, hypromellose, talcum and magnesium stearate. In order to prevent the pellets from sticking together at elevated temp-erature, a second coating containing a film-forming polymer is applied.

EP 0 520 119 A1 discloses diclofenac pellets coated with a membrane layer that contains Eudragit NE, talcum, magnesium stearate, polysorbate and silicone antifoam emulsion.

“Aqueous polyacrylate dispersions as coating materials for sustained and enteric release systems”, D. Wouessidjewe, S.T.P. Pharma Sciences 7 (6), 1997, pp. 469-475, describes coating with Eudragit NE30D. Eburnamonine-containing cores (diameter about 1000 m) are sprayed with an aqueous dispersion of Eudragit NE30D and 10% talcum as separating agent. Despite the use of talcum, Eudragit NE30D is observed to have a high level of stickiness. In order to prevent the pellets from sticking together during spraying with the Eudragit dispersion, a discontinuous spraying procedure is used in which the pellets are alternately sprayed and dried. The processing time on a laboratory scale is more than 9 hours, that is to say the procedure is very time-consuming.

In the literature, talcum is described as a known anti-sticking agent for aqueous poly(meth)acrylate dispersions. However, talcum very rapidly sediments in an aqueous suspension, that is to say the coating agent dispersions must be freshly prepared shortly before use and stirred constantly during spraying. Magnesium stearate is used more rarely as anti-sticking agent. A disadvantage of magnesium stearate is that it floats in aqueous dispersions, with the result that a spraying process becomes complicated to carry out.

For processing Eudragit NE30D, the manufacturer (Röhm) recommends the use of glycerol monostearate or talcum, micronised talcum being used in an amount of up to 100%, based on the polymer mass. Attempts at coating active-ingredient-containing cores with Eudragit NE30D/talcum suspensions in fluidised bed apparatus of different machine configurations were unsuccessful. After application of only 20% of the polymer, the micropellets became stuck together, consequently resulting in breakdown of the process.

The problem of the invention is to provide a pharmaceutical active-ingredient-containing formulation for oral administ-ration, preferably water-based, having improved process-ability, which has a low degree of stickiness, high mechanical strength and reproducibility during the processing-procedure and which in addition does not require further coatings or stabilizers such as surfactants or antifoams. A further objective is to provide a process for the prep-aration of coated formulations, such as pellets or tablets, wherein the preparation is to be cost-effective and time-saving. The coated formulations can have a modified release profile for the active ingredient(s), especially constant release (zero-order).

The problem underlying the invention is solved in accordance with an embodiment by a pharmaceutical active-ingredient-containing formulation for oral administration which is coated with a single coating of a film-forming polymer, the coating comprising a mixture of at least two separating agents and no stabilizer.

In the formulation according to the invention, the coating need not contain surfactant or antifoam as stabilizer.

Furthermore, in the formulation according to the invention the film-forming polymer can be distinguished by the fact that it can be provided in the form of a water-based dispersion.

Furthermore, in the formulation according to the invention the film-forming polymer can be a mixture of film-forming polymers.

The formulation according to the invention can also be provided with a polyacrylate as film-forming polymer.

Furthermore, in the formulation according to the invention the polyacrylate can be a polymer based on acrylic acid, methacrylic acid, acrylic acid esters and/or methacrylic acid esters, especially Eudragit and/or Kollicoat.

Furthermore, in the formulation according to the invention the mixture having the at least two separating agents can comprise
at least one separating agent that floats in pure water, and
at least one separating agent that sinks in pure water or dissolves therein.

Whether or not a separating agent floats in pure water need not depend solely on whether or not the density of the separating agent is greater than that of water. For example, magnesium stearate has a true density of 1.09 g/cm³, but floats in water.

Furthermore, in the formulation according to the invention, the mixture having the at least two separating agents can comprise
at least one fatty acid salt as separating agent and
at least one silicate from the group composed of double chain silicates and layer silicates as separating agent.

Furthermore, in the formulation according to the invention the mixture can comprise as floating separating agent or as fatty acid salt an alkali metal salt and/or an alkaline earth metal salt and/or an aluminium salt of a fatty acid.

Furthermore, in the formulation according to the invention the mixture can comprise sodium, potassium, magnesium and/or calcium behenate as alkali metal or alkaline earth metal salt of a fatty acid.

Furthermore, in the formulation according to the invention the mixture can comprise sodium, potassium, magnesium, calcium and/or aluminium stearate as alkali metal, alkaline earth metal or aluminium salt of a fatty acid.

Furthermore, in the formulation according to the invention the mixture can comprise a magnesium salt of caprylic acid, capric acid, lauric acid and/or palmitic acid as alkaline earth metal salt of a fatty acid.

Furthermore, in the formulation according to the invention the content of floating separating agent or of fatty acid salt can be from 5 to 40% by weight, preferably from 10 to 30% by weight, in each case based on the dry weight of the film-forming polymer.

Furthermore, in the formulation according to the invention the mixture can comprise a layer silicate as sinking separating agent or as silicate.

Furthermore, in the formulation according to the invention the mixture can comprise talcum, kaolinite, pyrophyllite, attapulgite, sepolite, muscovite, montmorillonite, bentonite and/or vermiculite as layer silicate.

Furthermore, in the formulation according to the invention the content of sinking separating agent or of silicate can be from 20 to 60% by weight, preferably from 30 to 50% by weight, in each case based on the dry weight of the film-forming polymer.

Furthermore, the formulation according to the invention can be in the form of active-ingredient-containing cores provided with the coating, which are capsules, tablets, pellets, granules, minitablets or micropellets.

Furthermore, the formulation according to the invention can be in the form of cores provided with the coating, which are active ingredient crystals.

Furthermore, in the formulation according to the invention, an active-ingredient-containing core in the form of a pellet or micropellet can comprise an inert core, an active-ingredient-containing core especially being constituted by an inert core with an active-ingredient-containing coating.

Furthermore, in the formulation according to the invention the micropellets can be provided as multiple-unit-dosage form, especially in the form of tablets or in capsules.

Furthermore, in the formulation according to the invention the pellets, granules or minitablets can be provided as multiple-unit-dosage form, especially in capsules.

Furthermore, in the formulation according to the invention the multiple-unit-dosage form can in turn be provided with a coating according to the invention.

Furthermore, in the formulation according to the invention the multiple-dosage form can be a capsule, especially a soft gelatin capsule.

Furthermore, in the formulation according to the invention the active ingredient can be provided in admixture with pharmaceutically acceptable auxiliaries, especially with customary auxiliaries.

Furthermore, in the formulation according to the invention the active ingredient can be provided in admixture with surfactants, especially non-ionic or ionic surface-active substances, or can be free of surfactants.

Furthermore, a formulation according to the invention can be provided with a readily water-soluble active ingredient, preferably with a solubility of more than 300 g/l aqueous solution.

For example, the formulation according to the invention can be provided with metoprolol or a salt thereof as active ingredient, especially metoprolol succinate.

In accordance with a further embodiment, the problem underlying the invention is solved according to the invention by an aqueous dispersion for the preparation of a coating for a pharmaceutical active-ingredient-containing formulation for oral administration, the dispersion having a content of a film-forming polymer and of at least two separ-ating agents and being free of stabilizers, wherein
at least one separating agent that floats in pure water is present in an amount of from 5 to 40% by weight, and
at least one separating agent that sinks in pure water is present in an amount of from 20 to 60% by weight,
   in each case based on the polymer dry weight.

Furthermore, according to a further embodiment the problem underlying the invention is solved according to the invent-ion by an aqueous dispersion for the preparation of a coating for a pharmaceutical active-ingredient-containing formulation for oral administration, the dispersion having a content of a film-forming polymer and of at least two separ-ating agents and being free of stabilizers, wherein
at least one fatty acid salt is present as separating agent in an amount of from 5 to 40% by weight, and
at least one silicate from the group composed of double chain silicates and layer silicates is present in an amount of from 20 to 60% by weight, in each case based on the polymer dry weight.

According to the invention, the dispersion can comprise no surfactant or antifoam as stabilizer,
in particular no non-ionic surfactant, especially no polysorbate, sorbitan monoisostearate, sorbitan mono-laurate, sorbitan monopalmitate, sorbitan monostearate, sorbitan monooleate, sorbitan sesquioleate, sorbitan trioleate, glyceryl monostearate, glyceryl monooleate and/or polyvinyl alcohol,
in particular no anionic surfactant, especially no sodium docusate and/or sodium lauryl sulfate,
in particular no cationic surfactant, especially no benzalkonium chloride, benzethonium chloride and/or cetrimide,
in particular no silicone-based antifoam and/or
in particular no glycerol, sorbitol and/or PEG deriva-tive as antifoam.

Finally, according to a further embodiment the problem underlying the invention is solved according to the invention by a process for the preparation of a pharma-ceutical active-ingredient-containing formulation, wherein a formulation that is as yet uncoated is provided with a coating using a dispersion according to the invention.

Surprisingly, therefore, it has been found inter alia that good processibility of aqueous polymer dispersions for the coating of cores (e.g. pellets or tablets) can be obtained when a mixture of at least two separating agents is added to the aqueous dispersions of the polymer(s). In that procedure, at least one separating agent can be an alkali metal, alkaline earth metal or aluminium salt of a fatty acid and at least one further separating agent can be a layer silicate. The tendency of the polymer(s) to stick together is reduced by the use of a mixture of at least two separating agents. Mixing together at least two separating agents of very different density evidently produces a density approximating that of water. In water, neither sedimentation of the layer silicate nor foam formation of the fatty acid salt occurs. It is unnecessary to use surfactants or antifoams.

By the use of two separating agents, the active-ingredient-containing cores are lipophilised, so that release can be slowed down and thus almost zero-order kinetics can be achieved.

When active-ingredient-containing cores are coated in accordance with the invention using aqueous polymer dispers-ions, the process is more cost-effective than procedures in which organic solvents are used for processing the polymer(s). Expensive, explosion-protected systems for coating active-ingredient-containing cores, as is the case with processes that are carried out using organic solvents, as well as the expensive disposal of such solvents are unnecessary. In addition, the preparation process according to the invention is time-saving, because the coating of the active-ingredient-containing cores can be effected using a mixture of at least two separating agents with a high spraying rate and without intermediate drying steps.

The invention therefore relates inter alia to formulations such as tablets or pellets having a single film coating, the film coating being applied from an aqueous dispersion of the film-forming polymer. Coating with the aqueous polymer dispersion is effected according to the invention using a mixture of at least two separating agents, especially an alkali metal or alkaline earth metal salt of a fatty acid and a layer silicate. The film coating is free of stabilizers such as surfactants or antifoams.

The invention therefore describes inter alia a film-forming system for the preparation of modified-release formulations with a single coating comprising:
a water-based polymer dispersion,
at least one fatty acid salt as separating agent, such as, for example, alkali metal or alkaline earth metal salts of fatty acids,
and at least one layer silicate as further separating agent,
   the at least two separating agents being mixed together and the resulting mixture being added to the polymer dispersion.

Using the film-forming system according to the invention, active-ingredient-containing cores can be coated with a single polymer-containing layer. The cores can comprise a pharmacologically effective substance and optionally one or more pharmaceutically acceptable auxiliaries.

Active ingredients having good solubility in water are especially preferred, because sustained release can be achieved only with difficulty by other methods. The solubility of the active ingredient in water is preferably more than 300 g/l.

Examples of water-soluble active ingredients which may be mentioned are: beta-blockers, such as metoprolol, biso-prolol; opioids, such as tramadol, morphine, oxycodon or hydrocodon. The active ingredients can be used in the form of stereoisomers or pharmaceutically acceptable salts, hydrates and solvates as well as in the form of derivatives. It is also possible to use combinations of two or more active ingredients. Preference is given to the use of meto-prolol or salts thereof such as tartrate, succinate, fumar-ate, benzoate or sorbate. The S-enantiomer of metoprolol or the benzoate or sorbate salt thereof can likewise be used. Special preference is given to the use of metoprolol succinate.

The active-ingredient-containing cores can be in the form of tablets, pellets, minitablets, granules or micropellets. The coated pellets or minitablets can be filled into capsules. The coated micropellets can be processed further to form tablets or capsules, that is to say multiple-unit-dosage forms. Active ingredient crystals and capsules, for example soft gelatin capsules, can also be coated with the film-forming system according to the invention.

The coated active-ingredient-containing cores exhibit modified release. Preferably, the active ingredient is released over a relatively long period of time, for example over from 10 to 24 hours.

### Active-Ingredient-Containing Cores:

Carriers and cores for the coatings can be capsules, tablets, granules, pellets or crystals. The size of granules, pellets or crystals can be between 0.01 and 2.5 mm, and that of tablets can be between 2.5 and 30.0 mm. The active ingredient content can vary within wide limits depending upon the active ingredient used and the desired rate of release. For example, the active ingredient content can be in the range of from 0.1 to 98% by weight, prefer-ably from 50 to 80% by weight, based on the total weight of the core.

The active-ingredient-containing core can be an active ingredient pellet or active ingredient granules, which contain(s) the active ingredient(s) and pharmaceutically customary auxiliaries. For that purpose, active ingred-ient(s) and auxiliaries are granulated together.

The active-ingredient-containing core in the form of pellets or micropellets can contain an “inert core” which is covered with an active-ingredient-containing layer. The “inert core” can consist of a water-insoluble material, for example glass, cellulose (e.g. microcrystalline cellulose), oxides and/or organic polymers. As organic polymers there are suitable polypropylene or polyethylene. It can also be composed of water-soluble material, such as inorganic salts, sugar or nonpareils. Such “inert cores” can have a diameter of from 10 to 2000 μm, preferably from 50 to 500 μm.

The inert core material can be coated with the active ingredient(s) in the form of crystals, agglomerates, etc. The active ingredient coating of the inert cores can be effected, for example, using granulation or spray-coating. The size of the active-ingredient-containing cores is from 200 to 2000 μm, preferably from 200 to 800 μm.

Prior to the coating of the inert core material, the active ingredient(s) can be mixed with auxiliaries, for example binders, surfactants, disintegrants and/or other pharma-ceutically acceptable auxiliaries. As binders there can be used celluloses, such as hydroxypropylmethylcellulose, hydroxypropylcellulose or sodium carboxymethylcellulose, polyvinylpyrrolidone, sugar and/or starch. Suitable surfactants are non-ionic or ionic surface-active sub-stances, such as, for example, sodium lauryl sulfate.

The active-ingredient-containing core can be a tablet or minitablet (diameter smaller than 4 mm). Such cores can contain, in addition to the active ingredient, further pharmaceutical auxiliaries such as carrier materials, fillers, binders, humectants, disintegration promoters, dis-integrants, lubricants, flow-regulators, mould release agents, preservatives, flavourings and/or colour pigments. Customary preparation processes are direct compression or compression of dry, moist or sinter granules.

### Coating:

The active-ingredient-containing cores can be coated with a single layer which contains one or more film-forming polymers and at least two separating agents.

Polyacrylates are suitable as water-based film-forming polymers.

The term “polyacrylate” denotes, for example, copolymers having two or more monomers such as acrylic acid, meth-acrylic acid, acrylate esters or methacrylate esters, such as, for example, aminoalkyl esters or alkyl esters, espe-cially methyl, ethyl, propyl and butyl esters, as well as hydroxylated acrylic or methacrylic acid esters. Examples of suitable film-forming polyacrylates are poly-ethyl acrylate-methyl methacrylates, poly-ethyl acrylate methacrylic acid, polymethacrylic acid methyl methacrylates and/or copolymers of acrylic and methacrylic acid esters having quaternary ammonium groups.

Polyacrylates are obtainable from Röhm under the trade name “Eudragit”. According to the invention, Eudragit NE, Eudragit RL, Eudragit RS, Eudragit L, Eudragit S, Eudragit FS or mixtures thereof can be used. Preference is given to the use of Eudragit NE30D.

Eudragit NE30D is poly-ethyl acrylate-methyl methacrylate in the form of a 30% strength aqueous dispersion, having a ratio of copolymers of 2:1. Eudragit NE exhibits a film-formation temperature of ˜5° C. (minimum). The copolymer has a neutral character and is water-insoluble over the entire pH range of the digestive tract. Eudragit NE exhibits pH-independent permeability. The coating of an active-ingredient-containing core with Eudragit NE accordingly results in diffusion-controlled retardation of the active ingredient release, because the Eudragit swells in water. Addition of plasticizers is not required for processing Eudragit NE30D.

A similar dispersion to Eudragit NE30D is Kollicoat EMM30D from BASF.

Eudragit NE40D is an aqueous dispersion with a 40% polymer content.

Eudragit RL and RS are copolymers of acrylic and methacrylic acid esters having a low content of quaternary ammonium groups, more specifically poly(ethyl acrylate-methyl meth-acrylate-trimethylammonium ethylmethyl methacrylate chlor-ide). The ratio of copolymers is 1:2:0.2 in the case of Eudragit RL and 1:2:0.1 in the case of Eudragit RS. The copolymers are water-insoluble over the entire pH range of the digestive tract and exhibit pH-independent permeability.

Eudragit RS, a weakly cationic hydrophilic polymethacrylate, requires an addition of from 10 to 20% plasticizer in order to lower the film-formation temperature to below 20° C. As plasticizers there are suitable triethyl acetyl citrate, diethyl sebacate, dibutyl sebacate, diethyl phthalate, dibutyl phthalate, triacetin, 1,2-propylene glycol, poly-ethylene glycol 6000 and especially triethyl citrate. Eudragit RS30D is in the form of a 30% aqueous dispersion.

Eudragit RL 30D requires an addition of about 20% plasticizer. As plasticizer there are suitable triethyl acetyl citrate, diethyl sebacate, triacetin, 1,2-propylene glycol and especially triethyl citrate.

Eudragit FS30D is a 30% strength dispersion containing a copolymer of 65% by weight methyl acrylate, 25% by weight methyl methacrylate and 10% by weight methacrylic acid.

Eudragit L30D55 is a 30% strength aqueous dispersion of a copolymer of anionic character based on methacrylic acid and ethyl acrylate. The ratio of the free carboxy groups to the ester groups is about 1:1. Eudragit L30D55 is suitable for enteric coatings and is soluble in intestinal fluid from pH=5.5. Eudragit L30D55 requires an addition of about from 10 to 15% plasticizer. Suitable plasticizers are triethyl citrate and polyethylene glycol.

Eudragit S is a copolymer having anionic character based on methacrylic acid and methyl methacrylate. The ratio of the free carboxy groups to the ester groups is about 1:2. Eudragit S is suitable for enteric coatings and is soluble in intestinal fluid from pH=7. The copolymer exhibits pH-dependent retardation. Eudragit S requires an addition of plasticizer, for example triethyl citrate or polyethylene glycol.

The content of polymer(s) can be from 40 to 90% by weight, based on the total coating. A content of from 60 to 70% by weight is preferred. The polymer(s) can be used together with a mixture of two or more separating agents, wherein at least one separating agent can be a fatty acid salt and at least one further separating agent can be a layer silicate.

As a fatty acid salt there are suitable, for example, alkali metal, alkaline earth metal or aluminium salts of fatty acids, such as sodium, potassium, magnesium, calcium or aluminium stearate, or sodium, potassium, magnesium or calcium behenate or magnesium salts of caprylic acid, capric acid, lauric acid or palmitic acid.

As layer silicates there are suitable talcum, kaolinite, pyrophyllite, attapulgite, sepiolite, muscovite, mont-morillonite, bentonite and/or vermiculite.

The content of one or more layer silicates can be from 20 to 60% by weight, based on the dry weight of the polymer(s). A content of from 30 to 50% by weight is preferred.

The content of one or more fatty acid salts can be from 5 to 40% by weight, based on the dry weight of the polymer(s). A content of from 10 to 30% by weight is preferred.

As further auxiliaries there can be used, for example, hydrophilic components (e.g. Aerosil or polyethylene glycols).

An addition of stabilizers, such as surfactants (e.g. non-ionic surfactants such as polysorbate, sorbitan mono-isostearate, sorbitan monolaurate, sorbitan monopalmitate, sorbitan monostearate, sorbitan monooleate, sorbitan sesqui-oleate, sorbitan trioleate, glyceryl monostearate, glyceryl monooleate, polyvinyl alcohol or anionic surfactants such as sodium docusate, sodium lauryl sulfate or cationic surfact-ants such as benzalkonium chloride, benzethonium chloride or cetrimide) or antifoams (e.g. silicone-based foam prevention agents such as polydimethylsiloxanes, Simethicone®, Dimethi-cone®, silicone emulsion or glycerol, sorbitol or PEG derivatives, is not required.

The polymers can be processed in the form of aqueous dispersions. There is no need to add organic water-miscible solvents such as lower alcohols, for example ethanol, propanol, isopropanol.

The layer thickness of the polymer coating is preferably from 25 to 75 μm.

When pellets are coated with the polymer dispersion according to the invention, the diameter of the coated pellets can be from 10 to 2000 μm. Micropellets are understood to be pellets having a diameter of less than 1000 μm. The coated micropellets according to the invention preferably have a diameter of from 300 to 800 μm.

### Process:

In the process according to the invention for the preparation of coated formulations, in a first step at least one fatty acid salt and at least one layer silicate can be mixed together. The mixing can take place at room temperature using mixing apparatus such as free-fall or ploughshare mixers, for example in a Turbula or Lödige mixer. The separating agent mixture can be added, with stirring, to an aqueous suspension of the polymer(s). It is also possible to add further auxiliaries. The polymer suspension so obtained can be applied to the active-ingredient-containing cores by spraying. The spray applic-ation of the aqueous polymer suspension can be carried out using “coating pans”, fluidised bed, Accela-cota, dip tube or dip blade processes or pan coating.

The spraying of tablets, minitablets or capsules can be carried out in a fluidised bed apparatus, a sugar-coating pan or a film-coating system having a perforated drum, air supply/removal means and spraying device.

For spraying active ingredient crystals, granules, pellets or micropellets it is possible to use fluidised bed apparatus of the “top spray”, “Wurster bottom spray” or “tangential spray” type (see also “Air suspension coating for multiparticulates”, D. Jones, Drug Development and Industrial Pharmacy, 20(20), 1994, pp. 3175-32.06).

For micropellets, special preference is given to the use of a fluidised bed apparatus from Glatt having a Wurster insert. A nozzle diameter of from 0.8 to 2.0 mm, a spraying rate of from 20 to 600 ml/min, a spraying pressure of from 1.0 to 2.7 bar as well as a product temperature of from 22° C. to 26° C. during the spraying procedure are advantageous. Drying of the micropellets can be effected in a fluidised bed apparatus, preferably at a product temperature of from 25 to 30° C. After sieving, flowable micropellets having a uniform particle size distribution are obtained.

### Further Processing to Form Capsules:

The coated, active-ingredient-containing pellets, micro-pellets, granules or minitablets can be filled into capsules. Suitable capsules are hard or soft gelatin capsules.

### Further Processing to Form Tablets:

In order to obtain a multiple-unit-dosage form in the form of a tablet it is possible for coated, active-ingredient-containing micropellets to be mixed with auxiliaries and compressed to form tablets.

Suitable auxiliaries for tablet preparation are:
fillers such as cellulose and/or cellulose derivatives (e.g. microcrystalline cellulose), sugar (e.g. lactose, glucose, saccharose), sugar alcohols (e.g. mannitol, sorbitol), starch (e.g. potato, wheat, corn and/or rice starch),
lubricants such as magnesium stearate, calcium stearate, stearic acid, hydrogenated vegetable oils and/or talcum,
flow-regulators such as highly dispersed silicon dioxide,
disintegrants such as starch and starch derivatives (sodium carboxymethyl starch), cross-linked polyvinyl-pyrrolidone, unmodified or modified cellulose (e.g. sodium carboxymethylcellulose, cross-linked carboxymethylcellulose) and/or alginates.

The tablets can be coated with a film-forming material in order to obtain a smooth surface or to increase the stability of the tablet during packaging and transport. Such a tablet coating can comprise, for example, additives such as “anti-tacking” materials or colourings.

The content of micropellets can be a maximum of 70% of the total tablet weight. Preference is given to a content of from 25 to 55%.

### Release Profile:

In vitro release studies for the described formulations were carried out using a USP standard apparatus with simulated gastric fluid (pH=1.2) or in buffer medium (pH=6.8).

The invention is described in greater detail by the following Examples, but the scope of the invention is not limited thereby.

### EXAMPLE 1

The following substances are used for the preparation of morphine sulfate tablets:

| | | Weight |
|---|---|---|
| Constituents | Percent | (mg/tablet) |
| Morphine sulfate | 2.3 | 10.0 |
| Cellulose pellets | 21.0 | 90.0 |
| Eudragit NE30D | 10.3* | 44.5* |
| Talcum | 6.0 | 25.7 |
| Calcium stearate | 4.1 | 17.6 |
| Lactose | 49.3 | 212.0 |
| Stearic acid | 1.0 | 4.2 |
| Sodium carboxymethylcellulose | 3.0 | 12.9 |
| Highly dispersed silicon | 0.5 | 2.1 |
| dioxide | | |
| Hydroxypropylmethylcellulose | 2.1 | 9.0 |
| PEG 8000 | 0.2 | 1.0 |
| Iron oxide yellow | 0.2 | 1.0 |
| Total | 100.0 | 430.0 |

*the amount of film-coating dry substance is given

### Preparation:

The cellulose pellets are first sprayed with an aqueous solution of morphine sulfate. The active-ingredient-containing cores are then coated with a layer of Eudragit NE3.0D/talcum/calcium stearate. The coated pellets are then mixed with lactose (filler), stearic acid (lubricant), sodium carboxymethylcellulose (disintegrant) and highly dispersed silicon dioxide (flow-regulator) and compressed to form tablets. The tablets are then film-coated with a water-soluble HPMC film-coating which contains PEG 8000 as plasticizer and iron oxide as colour pigment.

### EXAMPLE 2

The following substances are used for the preparation of metoprolol succinate tablets.

| | | Weight |
|---|---|---|
| Constituents | Percent | (mg/tablet) |
| Metoprolol succinate | 15.8 | 95.0 |
| Sugar pellets | 27.0 | 162.2 |
| Eudragit NE30D | 13.4* | 80.2* |
| Talcum | 3.0 | 18.3 |
| Magnesium stearate | 0.7 | 4.3 |
| Microcrystalline cellulose | 36.5 | 219.0 |
| Crospovidone | 3.4 | 20.2 |
| Highly dispersed silicon | 0.2 | 0.8 |
| dioxide | | |
| Total | 100.0 | 600.0 |

*the amount of film-coating dry substance is given

### Preparation:

The sugar pellets are first sprayed with an aqueous solution of metoprolol succinate. The active-ingredient-containing cores are then coated with a layer of Eudragit NE30D/talcum/magnesium stearate. The coated pellets are compressed together with further auxiliaries to form tablets.

### Comparison of Release Profiles

Comparison of the release profile of a metoprolol succinate tablet which contains metoprolol-succinate-containing cores coated with Eudragit NE3.0D, magnesium stearate and talcum, with a corresponding formulation in which only talcum was used as separating agent.

Apparatus for determining active ingredient release:

| | |
|---|---|
| Medium: | KH₂PO₄ buffer pH 6.8, 900 ml, paddle |
| Temperature: | 37° C. |
| Stirring speed: | 100 rev/min |

| | Coating with Eudragit | Coating with |
|---|---|---|
| | NE30D/talcum/magnesium | Eudragit NE30D/ |
| | stearate | talcum |
| | Active ingredient | Active ingredient |
| Time (min) | released (%) | released (%) |
| 120 | 18 | 5 |
| 360 | 44 | 25 |
| 600 | 65 | 60 |
| 840 | 80 | 78 |
| 1200 | 95 | 85 |

As can be seen from the Table, when talcum and magnesium stearate are used as separating agent, a zero-order release profile is obtained. When talcum alone is used, a first-order release profile is obtained.

### EXAMPLE 3

The following substances are used for the preparation of bisoprolol fumarate tablets.

| | | Weight |
|---|---|---|
| Constituents | Percent | (mg/tablet) |
| Bisoprolol fumarate | 2.4 | 10.0 |
| Cellulose pellets | 46.3 | 190.0 |
| Eudragit NE30D | 10.2* | 42.0* |
| Bentonite | 4.9 | 20.0 |
| Aluminium stearate | 2.0 | 8.0 |
| Microcrystalline cellulose | 21.1 | 86.5 |
| Lactose | 9.1 | 37.1 |
| Sodium carboxymethylcellulose | 3.0 | 12.3 |
| Highly dispersed silicon | 1.0 | 4.1 |
| dioxide | | |
| Total | 100.0 | 410.0 |

*the amount of film-coating dry substance is given

### Preparation:

The cellulose pellets are first sprayed with an aqueous solution of bisoprolol fumarate. The active-ingredient-containing cores are then coated with a layer of Eudragit NE30D/bentonite/aluminium stearate. The coated pellets are compressed together with further auxiliaries to form tablets.

### EXAMPLE 4

The following substances are used for the preparation of tramadol hydrochloride capsules.

| | | Weight |
|---|---|---|
| Constituents | Percent | (mg/capsule) |
| Tramadol hydrochloride | 28.2 | 100.0 |
| Sugar pellets | 28.2 | 100.0 |
| Eudragit NE30D | 18.0* | 64.0* |
| Talcum | 3.6 | 12.8 |
| Calcium behenate | 0.9 | 3.2 |
| Hard gelatin capsule | 21.3 | 76.0 |
| Total | 100.0 | 356.0 |

*the amount of film-coating dry substance is given

### Preparation:

The sugar pellets are first sprayed with an aqueous solution of tramadol hydrochloride. The active-ingredient-containing cores are then coated with a layer of Eudragit NE30D/talcum/calcium behenate. The coated pellets are filled into hard gelatin capsules.

### EXAMPLE 5

The following substances are used for the preparation of oxycodon hydrochloride capsules.

| | | Weight |
|---|---|---|
| Constituents | Percent | (mg/capsule) |
| Oxycodon hydrochloride | 3.1 | 10.0 |
| Microcrystalline cellulose | 47.8 | 155.7 |
| Magnesium stearate | 0.5 | 1.7 |
| Aerosil | 0.8 | 2.6 |
| Eudragit NE30D | 15.3* | 50.0* |
| Kaolin | 6.1 | 20.0 |
| Magnesium stearate | 3.1 | 10.0 |
| Hard gelatin capsule | 23.3 | 76.0 |
| Total | 100.0 | 326.0 |

*the amount of film-coating dry substance is given

### Preparation:

Oxycodon hydrochloride is compressed together with micro-crystalline cellulose, Aerosil and magnesium stearate to form minitablets. The minitablets are then coated with a layer of Eudragit NE30D/kaolin/magnesium stearate. The coated minitablets are filled into hard gelatin capsules.

1. Pharmaceutical active-ingredient-containing formulation for oral administration which is coated with a single coating of a film-forming polymer, the coating comprising a mixture of at least two separating agents and no stabilizer.

2. Formulation according to claim 1, wherein the coating does not contain surfactant or antifoam as stabilizer.

3. Formulation according to claim 1, wherein the film-forming polymer is characterised in that it can be provided in the form of a water-based dispersion.

4. Formulation according to at claim 1, wherein the film-forming polymer is a mixture of film-forming polymers.

5. Formulation according to at claim 1 having a polyacrylate as film-forming polymer.

6. Formulation according to claim 5, wherein the polyacrylate is a polymer based on acrylic acid, methacrylic acid, acrylic acid ester and/or methacrylic acid ester, especially Eudragit and/or Kollicoat.

7. Formulation according to claim 1, wherein the mixture having the at least two separating agents comprises
at least one separating agent that floats in pure water, and
at least one separating agent that sinks in pure water.

8. Formulation according to claim 1, wherein the mixture having the at least two separating agents comprises
at least one fatty acid salt as separating agent and
at least one silicate from the group composed of double chain silicates and layer silicates as separating agent.

9. Formulation according to claim 7, wherein the
mixture comprises as floating separating agent or as fatty acid salt an alkali metal salt and/or an alkaline earth metal salt and/or an aluminium salt of a fatty acid.

10. Formulation according to claim 9, wherein the mixture comprises sodium, potassium, magnesium and/or calcium behenate as alkali metal or alkaline earth metal salt of a fatty acid.

11. Formulation according to claim 9, wherein the mixture comprises sodium, potassium, magnesium, calcium and/or aluminium stearate as alkali metal, alkaline earth metal or aluminium salt of a fatty acid.

12. Formulation according to claim 9, wherein the mixture comprises a magnesium salt of caprylic acid, capric acid, lauric acid and/or palmitic acid as alkaline earth metal salt of a fatty acid.

13. Formulation according to claim 7, wherein the content of floating separating agent or of fatty acid salt is from 5 to 40% by weight, preferably from 10 to 30% by weight, in each case based on the dry weight of the film-forming polymer.

14. Formulation according to claim 7, wherein the mixture comprises a layer silicate as sinking separating agent or as silicate.

15. Formulation according to claim 14, wherein the mixture comprises talcum, kaolinite, pyrophyllite, attapulgite, sepolite, muscovite, montmorillonite, bentonite and/or vermiculite as layer silicate.

16. Formulation according to claim 7, wherein the content of sinking separating agent or of silicate is from 20 to 60% by weight, preferably from 30 to 50% by weight, in each case based on the dry weight of the film-forming polymer.

17. Formulation according to claim 1 in the form of active-ingredient-containing cores provided with the coating, which are capsules, tablets, pellets, granules, minitablets or micropellets.

18. Formulation according to claim 1 in the form of cores provided with the coating, which are 25 active ingredient crystals.

19. Formulation according to claim 17, wherein an active-ingredient-containing core in the form of a pellet or micropellet comprises an inert core, an active-ingredient-containing core especially being constituted by an inert core with an active-ingredient-containing coating.

20. Formulation according to claim 17, wherein the micropellets are provided as multiple-unit-dosage form, especially in the form of tablets or in capsules.

21. Formulation according to claim 17, wherein the pellets, granules or minitablets are provided as multiple-unit-dosage form, especially in capsules.

22. Formulation according to claim 20, wherein the multiple-unit-dosage form is in turn provided with a coating comprising a mixture of at least two separating agents and no stabilizer.

23. Formulation according to claim 20, wherein the multiple-dosage form is a capsule, especially a soft gelatin capsule.

24. Formulation according to claim 1, wherein the active ingredient is provided in admixture with pharmaceutically acceptable auxiliaries, especially with customary auxiliaries.

25. Formulation according to claim 1, wherein the active ingredient is provided in admixture with surfactants, especially non-ionic or ionic surface-active substances, or is free of surfactants.

26. Formulation according to claim 1 having a readily water-soluble active ingredient, preferably with a solubility of more than 300 g/l aqueous solution.

27. Formulation according to claim 1 with metoprolol or a salt thereof as active ingredient, especially metoprolol succinate.

28. Aqueous dispersion for the preparation of a coating for a pharmaceutical active-ingredient-containing formulation for oral administration according to claim 1, the dispersion having a content of a film-forming polymer and of at least two separating agents and being free of stabilizers, wherein
at least one separating agent that floats in pure water is present in an amount of from 5 to 40% by weight, and
at least one separating agent that sinks in pure water is present in an amount of from 20 to 60% by weight, in each case based on the polymer dry weight.

29. Aqueous dispersion for the preparation of a coating for a pharmaceutical active-ingredient-containing formulation for oral administration according to claim 1, the dispersion having a content of a film-forming polymer and of at least two separating agents and being free of stabilizers, wherein
at least one fatty acid salt is present as separating agent in an amount of from 5 to 40% by weight, and
at least one silicate from the group composed of double chain silicates and layer silicates is present in an amount of from 20 to 60% by weight,
in each case based on the polymer dry weight.

30. Dispersion according to claim 28, wherein the dispersion comprises no surfactant or antifoam as stabilizer,
in particular no non-ionic surfactant, especially no polysorbate, sorbitan monoisostearate, sorbitan monolaurate, sorbitan monopalmitate, sorbitan monostearate, sorbitan monooleate, sorbitan sesquioleate, sorbitan trioleate, glyceryl monostearate, glyceryl monooleate and/or polyvinyl alcohol,
in particular no anionic surfactant, especially no sodium docusate and/or sodium lauryl sulfate,
in particular no cationic surfactant, especially no benzalkonium chloride, benzethonium chloride and/or cetrimide,
in particular no silicone-based antifoam and/or in particular no glycerol, sorbitol and/or PEG derivative as antifoam.

31. Process for the preparation of a pharmaceutical active-ingredient-containing formulation according to claim 1, wherein a formulation that is as yet uncoated is provided with a coating using a dispersion.
